(19) United States
(12) Patent Application Publication
(10) Pub. No.: US 2015/0283739 A1
(43) Pub. Date: Oct. 8, 2015

(54) FIBRE-REINFORCED THERMOPLASTIC COMPONENT ASSEMBLY AND METHOD FOR PRODUCING SUCH A FIBRE-REINFORCED THERMOPLASTIC COMPONENT ASSEMBLY

(71) Applicant: BROSE FAHRZEUGTEILE GMBH & CO. KOMMANDITGESELLSCHAFT, COBURG, Coburg (DE)

(72) Inventors: Andrea Bauersachs, Ebersdorf (DE); Ulf Hartmann, Schweinfurt (DE)

(21) Appl. No.: 14/442,827

(22) PCT Filed: Nov. 13, 2013
(86) PCT No.: PCT/EP2013/073674

§ 371 Date: May 14, 2015

(30) Foreign Application Priority Data
Nov. 14, 2012 (DE) 10 2012 220 782.1

(51) Int. Cl. B29C 45/14; B32B 5/02; G01N 1/30; G01N 21/25; G01N 33/44; B32B 27/00; G01K 11/06

(52) U.S. Cl. CPC B29C 45/14; B32B 27/00; B32B 5/02; G01K 11/06; G01N 21/25; G01N 33/442; G01N 1/30; B29K 2101/12

(57) ABSTRACT

The invention relates to a method for producing a fibre-reinforced thermoplastic component assembly and to a fibre-reinforced thermoplastic component assembly, comprising: a fibre-reinforced thermoplastic component, which has a first side and a second side, opposite from the first side, at least one additional element of a thermoplastic material, which is arranged on the first side of the fibre-reinforced thermoplastic component, and at least one testing element of a thermoplastic material, which is visibly arranged on the first or second side of the fibre-reinforced thermoplastic component and is designed for testing the connection of the additional element to the fibre-reinforced thermoplastic component.

The invention relates to a fibre-reinforced thermoplastic component assembly, such as an organic sheet component, and to a method for producing a fibre-reinforced thermoplastic component assembly of this type.

A fibre-reinforced thermoplastic component assembly, such as an organic sheet component assembly, consists of a thermoplastic fibre/plastics material composite.

Fibre-reinforced thermoplastics are composite materials which consist of both reinforcing fibres for absorbing loads and a matrix material for ensuring fibre orientation and dimensional stability. For example, glass fibres, aramid fibres, carbon fibres, etc., as well as natural fibres, such as sisal, coconut, hemp, flax, etc. can be used in this case as fibrous materials. In this regard, a distinction is made between short, long and continuous reinforcing fibres, short fibres and long fibres mostly being injection moulded or extruded directly with the thermoplastic granules. Continuous reinforcing fibres are in turn generally processed as fibre strands, rovings, weft-knitted fabrics, woven fabrics or braided fabrics. A thermoplastic can be used as the matrix material.

To produce continuous-fibre-reinforced thermoplastic components in a profiled shape, winding technology, pultrusion or interval hot pressing can be used for example. Today, planar components are produced using the thermoforming method together with additional back injection or extrusion (for example injection moulding process). For this purpose, use is generally made of semi-finished products which are provided in the form of partly or completely pre-consolidated sheets and are referred to as organic sheets or thermoplastic prepregs. Textile fabrics consisting of hybrid yarn, in which the matrix is also fibrous in addition to the reinforcing fibre, can also be used as a semi-finished product.

The German utility model DE 20 2006 019 341 U1 describes an organic sheet structural component having a structure-stiffening plastics insert. Here, the plastics insert is back injected or at least partly encapsulated with a thermoplastic material such that the plastics insert and the thermoplastic material become integrally bonded to one another.

In organic sheet structures as an example of a fibre-reinforced thermoplastic component assembly, the strength of the connection between the rest of the organic sheet and an additional element, for example a rib, formed on the organic sheet was hitherto determined by means of a destructive test.

Against this background, the problem addressed by the present invention is that of disclosing a higher quality fibre-reinforced thermoplastic component assembly and of providing a method by which the quality of the thermoplastic component assembly can be ascertained.

This problem is solved according to the invention by a fibre-reinforced thermoplastic component assembly having the features of claim 1 and/or by a method having the features of claim 16.

The following is accordingly provided:
a fibre-reinforced thermoplastic component assembly comprising
a fibre-reinforced thermoplastic component having a first side and a second side opposite the first side,
at least one additional element made of a thermoplastic and arranged on the first side of the fibre-reinforced thermoplastic component, and
at least one testing element which is made of a thermoplastic, arranged on the first or second side of the fibre-reinforced thermoplastic component so as to be visible, and is designed for testing the connection between the additional element and the fibre-reinforced thermoplastic component.
A method for producing a fibre-reinforced thermoplastic component assembly, comprising the steps of: providing a fibre-reinforced thermoplastic component having
a first side and a second side opposite the first side;
forming, from a thermoplastic, at least one additional element on the first side of the fibre-reinforced thermoplastic component; and
forming, from a thermoplastic, at least one testing element on the first or second side of the fibre-reinforced thermoplastic component, wherein the at least one testing element is arranged on the fibre-reinforced thermoplastic component so as to be visible and is designed for testing the connection between the additional element and the fibre-reinforced thermoplastic component.

The testing element formed on the fibre-reinforced thermoplastic component assembly so as to be visible has the advantage that it allows the connection between the additional element and the fibre-reinforced thermoplastic component of the thermoplastic component assembly to be tested in a non-destructive manner, since conclusions can be drawn regarding the connection between the additional element and the organic sheet component from the degree of formation of the testing element and/or, when different colour thermoplastics are used, from the colouring of the testing element. The degree of formation of the testing element, for example a completely formed testing element or only a partly formed testing element, can be determined in a very simple and non-destructive manner by a visual check or by means of a camera. The same applies to the colour or colour mixing of the testing element. As a result, there is no need for a destructive test, for example a tension test or sectional image. Instead, it is possible according to the invention to provide, for example, a simple, non-destructive quality check for the batch production of fibre-reinforced, in particular continuous-fibre-reinforced, thermoplastic component assemblies.

Advantageous embodiments and developments of the invention are found in the additional dependent claims and in the description with reference to the figures of the drawings.

In one embodiment of the invention, the at least one testing element and/or the at least one additional element are formed on the fibre-reinforced thermoplastic component for example by means of injection moulding or by extrusion. As with injection moulding, extrusion allows the testing element and additional element to be produced in a very simple manner from a thermoplastic.

In another embodiment of the invention, at least one testing element is formed as a melt island. A melt island of this type can be integrated in a component and inspected in a particularly simple manner.

In a further embodiment of the invention, the at least one testing element is arranged on the at least one additional element as an extra portion. This allows the testing element to be directly joined to the additional element.

In another embodiment, a different colour thermoplastic is used in each case to form the at least one testing element, the at least one additional element and/or the thermoplastic component. On the basis of the colour change or colour gradient of the thermoplastic for the testing element, it can thus be established for example whether or not the thermoplastic of the thermoplastic component that is a different colour from that of the testing element has mixed with the plastics material of the testing element to a suitable extent. If there is no visible mixing of the plastics material of the thermoplastic component and that of the testing element, it can thus be concluded that the temperature of the thermoplastic component was not suitably controlled and therefore that the strength of the connection between the additional element and the thermoplastic component is insufficient.

According to a further embodiment of the invention, at least one testing element is formed as a functional element, the functional element being for example a rib, a mount, a guide, a reinforcement, a stiffener and/or a clip. As a result, in addition to the ability to inspect the connection between the additional element and the fibre-reinforced thermoplastic component, the testing element can also assume a function whereby it simultaneously acts as a functional element, in particular as a rib, a mount, a guide and/or a clip, etc.

In one embodiment of the invention, the testing element is arranged on the fibre-reinforced thermoplastic component assembly so as to be visible to a camera so that the testing element can be visually assessed by means of at least one camera. In this case, the camera images can, for example, be assessed by a user or automatically by means of a computer.

In a further embodiment of the invention, the testing element is arranged on the fibre-reinforced thermoplastic component assembly so as to be visible to the eye of a user so that the testing element can be assessed by means of a visual check. A visual check by a user is a particularly simple and reliable option for assessing the testing element and thus for drawing conclusions on the connection between the additional element and the fibre-reinforced thermoplastic component.

According to another embodiment, a plurality of testing elements having defined flow paths are provided on the fibre-reinforced thermoplastic component. It is thus possible, for example, to determine whether or not the connection between the additional element and the fibre-reinforced thermoplastic component is as intended not only in one region, but also, for example, over the entire part of the additional element.

In one embodiment of the invention, the additional element is for example a rib, a mount, a guide, a reinforcement, a stiffener and/or a clip. However, the invention is not limited to the above examples. Additional structures, for example profiles, can also be worked into the additional element, or the additional element can be integrally moulded to these structures, or said structures can be encapsulated by the additional element.

According to one embodiment of the invention, the fibre-reinforced thermoplastic component assembly is a fibre arrangement of glass fibres, aramid fibres, carbon fibres, sisal, hemp, coconut fibres, cotton fibres and/or flax, etc. In this regard, the fibre arrangement can form at least a woven fabric, a fibre strand, a roving, a weft-knitted fabric, a braided fabric, a non-woven fabric, a mat and/or a fibrous web. Besides the above-mentioned natural fibres, other natural fibres can also be used. In the same way, the invention is not limited to glass fibres, aramid fibres and carbon fibres either. In this context, short, long and/or continuous fibres can be used for the fibres.

In one embodiment of the invention, the fibre-reinforced thermoplastic component assembly comprises an organic sheet or a pre-consolidated Twintex® sheet as a fibre-reinforced thermoplastic component. An organic sheet and a Twintex® sheet have the advantage that the reinforcing fibres are already completely impregnated and consolidated and so only a short processing time and relatively low pressure are required for deformation.

In a further embodiment of the invention, the fibre-reinforced thermoplastic component is deformed before, during and/or after the step of forming at least one additional element and/or at least one testing element on the fibre-reinforced thermoplastic component. The deformation of the fibre-reinforced thermoplastic component together with the formation of the testing element and the additional element has the advantage that production times and costs can be reduced, since all the steps take place at once and only one mould is required.

The above embodiments and developments can be combined in any conceivable manner, as long as this is reasonable. Further possible embodiments, developments and uses of the invention also include combinations of features of the invention described previously or below with respect to the embodiments, even if not explicitly stated. In this context, a person skilled in the art will more particularly also add individual aspects as improvements or additions to the respective basic form of the present invention.

The present invention is explained below in greater detail with the aid of embodiments specified in the schematic figures in the drawings. These are as follows:

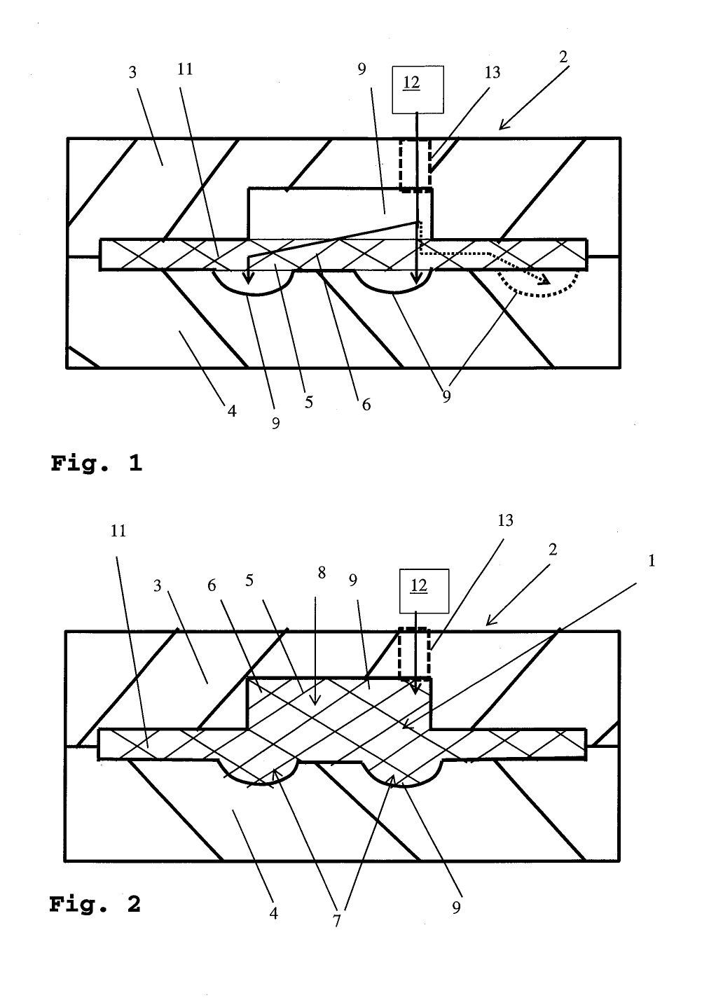

FIG. 1 is a sectional view of a mould having a fibre-reinforced thermoplastic component received therein, prior to plastics material being injected;

FIG. 2 is a sectional view of the mould and the fibre-reinforced thermoplastic component received therein, after plastics material has been injected;

FIG. 3 is a sectional view of a fibre-reinforced thermoplastic component assembly in which a testing element is formed only in part;

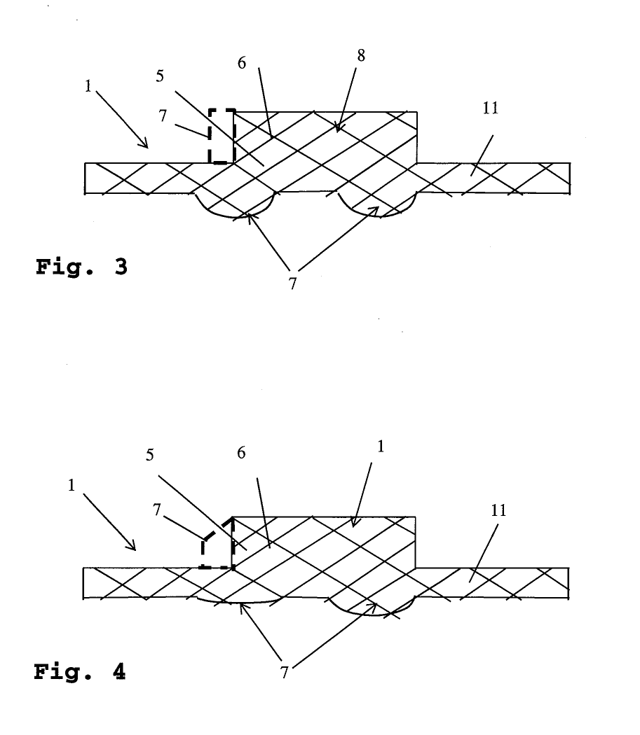

FIG. 4 is a sectional view of a fibre-reinforced thermoplastic component assembly in which a testing element is completely formed;

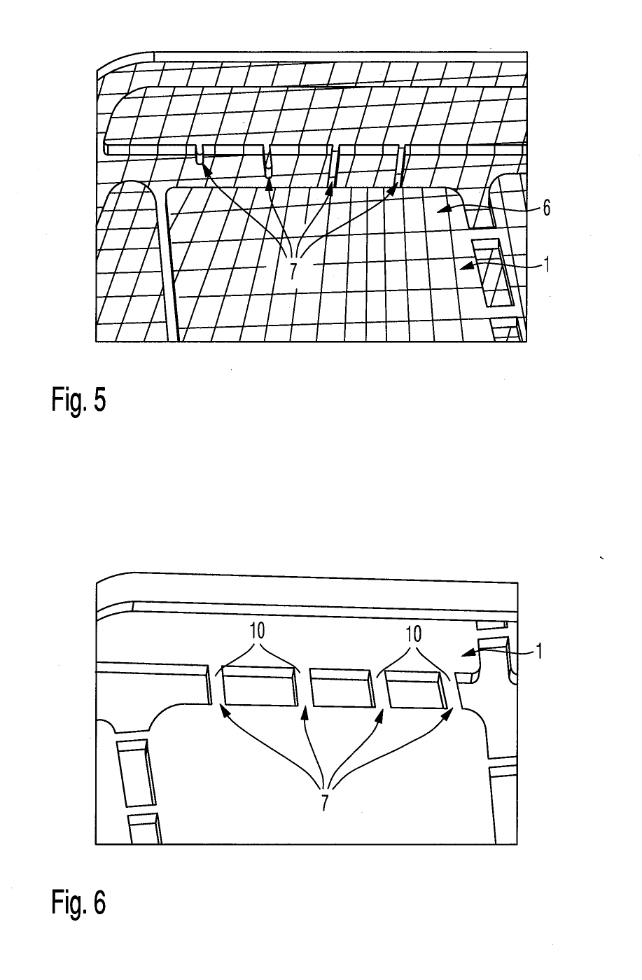

FIG. 5 is a perspective view of a fibre-reinforced thermoplastic component assembly in which testing elements could not be formed;

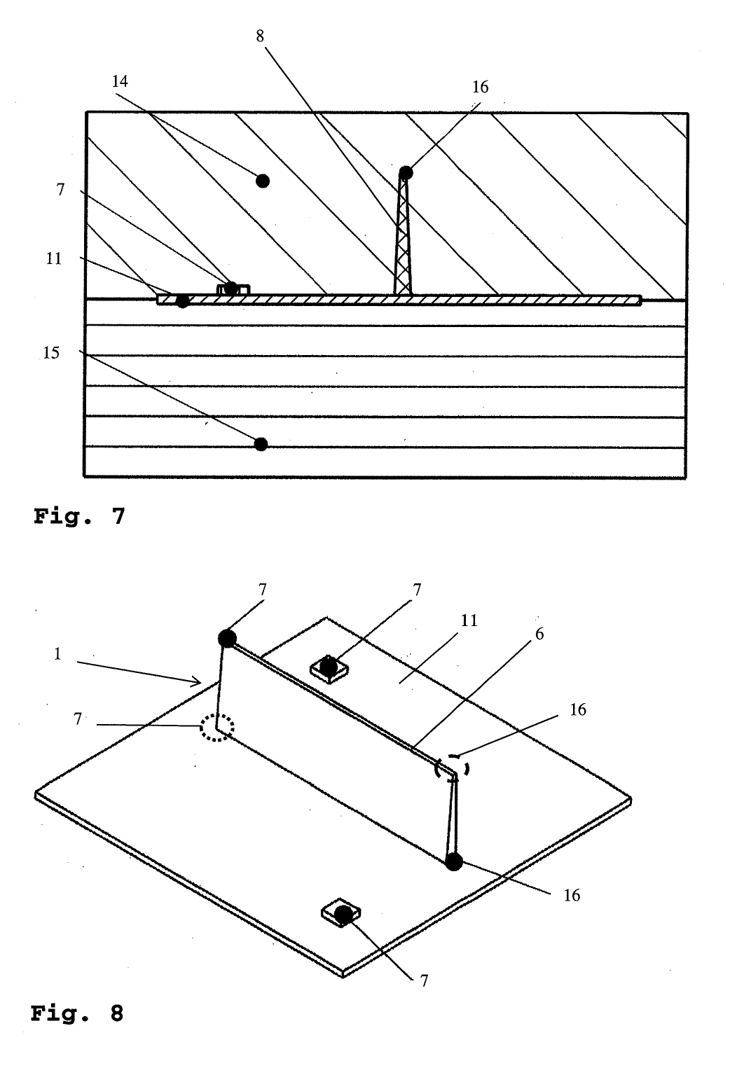

FIG. 6 is a perspective view of a fibre-reinforced thermoplastic component assembly in which testing elements could be completely formed;

FIG. 7 is a sectional view through a mould and a fibre-reinforced thermoplastic component received therein, after plastics material has been injected;

FIG. 8 is a perspective view of the finished thermoplastic component assembly according to FIG. 7; and

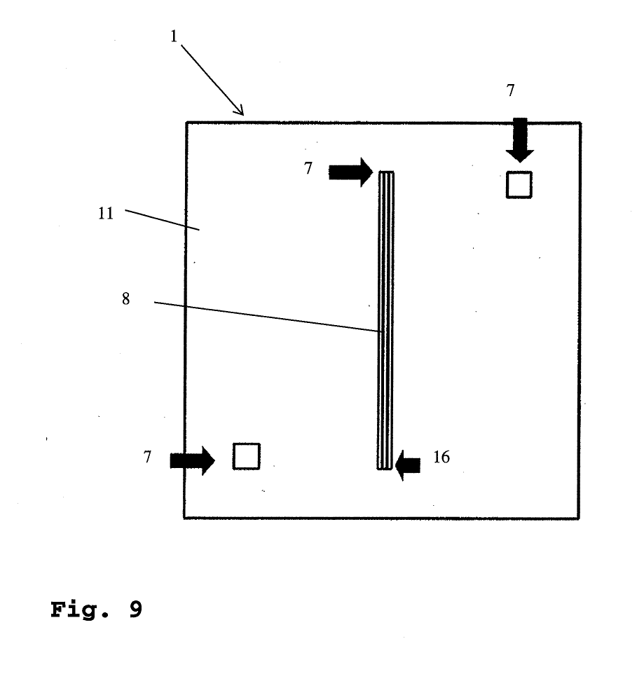

FIG. 9 is a plan view of the thermoplastic component assembly according to FIG. 8.

The accompanying drawings are intended to convey better understanding of the embodiments of the invention. They show embodiments of the invention and, in conjunction with the description, clarify the principles and concepts behind the invention. Other embodiments and many of the aforementioned advantages become apparent with respect to the drawings. The elements of the drawings are not necessarily shown true to scale in relation to each other.

In the figures in the drawing, same elements, features and components, or those serving the same function and having the same effect, are provided with same reference numerals in each case, unless otherwise specified.

### DESCRIPTION OF EMBODIMENTS

FIGS. 1 and 2 are both sectional views of a mould 2 having a fibre-reinforced thermoplastic component 11 placed therein for producing a fibre-reinforced thermoplastic component assembly 1 comprising at least one additional element 8 and at least one testing element 7. In this case, in FIG. 1 the fibre-reinforced thermoplastic component 11, for example a fibre-reinforced semi-finished product, is placed in the mould 2 so that an additional element 8 and, for example, two testing elements can then be integrally moulded thereto. FIG. 2 shows the mould 2 according to FIG. 1 once the additional element 8 and the two testing elements 7 have been integrally moulded to the fibre-reinforced thermoplastic component. In this case, the finished thermoplastic component assembly 1 as shown in FIG. 2 comprises the fibre-reinforced thermoplastic component 11 having the additional element 8 integrally moulded thereto and the two integrally moulded testing elements 7.

During production of a component assembly from a fibre-reinforced thermoplastic component such as an organic sheet, ribs or other additional elements 8 have to be pressed on or integrally moulded at a sufficiently high temperature in order to obtain a high-quality attachment. However, if the temperature of the fibre-reinforced thermoplastic component 11 is not sufficiently high or suitably controlled, this can lead to a significant drop in the strength of the connection between the additional elements 8, for example ribs, and the thermoplastic component 11 of the fibre-reinforced thermoplastic component assembly 1.

Destructive tests have hitherto been used to test whether the temperature of the fibre-reinforced thermoplastic component 11 was adequately controlled or whether the component was heated to a suitably high temperature, and whether the resultant strength of the connection between the ribs or other additional elements 8 and the fibre-reinforced thermoplastic component 11 was adequate. An example of a destructive test of this type is a tension test or a micrograph to inspect weld lines. With large components such as seat shells, a CT analysis can also only be carried out on sections thereof.

According to the invention, a fibre-reinforced thermoplastic component 11 is therefore formed having at least one additional testing element 7 which allows the connection between the additional element 8 and the fibre-reinforced thermoplastic component 11 to be tested in a non-destructive manner.

As shown in the embodiment in FIGS. 1 and 2, the fibre-reinforced thermoplastic component 11 is formed, for example, having at least one additional element 8, such as a rib. At least one testing element 7, or two testing elements 7 as shown in FIGS. 1 and 2, is formed on the fibre-reinforced thermoplastic component 11 to subsequently establish whether the temperature of the fibre-reinforced thermoplastic component 11 was adequately controlled or whether the component was heated to a suitably high temperature during production, and whether the rib as the additional element 8 is accordingly adequately integrally bonded to the fibre-reinforced thermoplastic component 11. In the process, the testing elements 7 are preferably arranged such that the injected or sprayed thermoplastic 12 has to cover a flow path towards the respective testing element 7 that is at least the same as or is larger than that towards the additional element.

The mould shown in FIGS. 1 and 2 is, for example, an injection mould 2 having first and second mould halves 3, 4 for injecting a thermoplastic 12. In this case, one mould half 3 has one cavity 9 for forming the additional element 8 and the other mould half 4 has two cavities 9 for forming the two testing elements 7 on the fibre-reinforced thermoplastic component 11. The cavity 9 for the additional element 8 and the two cavities 9 for the two testing elements 7 preferably have a defined shape for forming the additional element 8 and the two testing elements 7 with a predetermined or defined shape.

In this regard, the testing element 7 can be formed either as a melt island not having any additional function or as a functional element having an additional function, for example as a rib, reinforcement, stiffener, clip, guide, for example cable guide, and/or a mount, etc. The invention is not limited to the above examples for testing elements 7.

The testing element 7 in each case is used for testing the connection between the additional element 8 and the fibre-reinforced thermoplastic component 11 of the fibre-reinforced thermoplastic component assembly 1. The test is carried out on the basis of whether the testing element 7, for example having its predetermined or defined shape, has been completely formed or formed only in part or not formed at all on the fibre-reinforced thermoplastic component assembly 1. Conclusions can be drawn therefrom on for example the strength of the connection between the additional element 8 and the fibre-reinforced thermoplastic component 11 of the thermoplastic component assembly 1, as will be described in more detail below with reference to FIG. 3-6.

The semi-finished product or pre-shaped component is heated to the processing temperature of the plastics matrix and placed in the tool. When the components have thin walls, heating in the tool is also possible. The tool is then closed and the plasticised additive is injected. The hollow space in the additional element is filled and the material then has to flow further into the testing element. This only happens if the temperature of the organic sheet is still above the crystallisation limit.

As shown in the embodiment in FIGS. 1 and 2, the temperature of the fibre-reinforced thermoplastic component 11 received in the injection mould 2 is suitably controlled or said component is heated to a suitable processing temperature, at which a liquid thermoplastic 12 which has also been suitably heated is injected into the mould 2.

In this process, the injected thermoplastic 12 is, for example, the same as the thermoplastic of the plastics matrix of the fibre-reinforced thermoplastic component 11 and in this case can be the same colour as or a different colour from the plastics material of the plastics matrix of the thermoplastic component 11. Alternatively, it is also possible to inject a different thermoplastic which can be joined to the thermoplastic of the plastics matrix 5 of the fibre-reinforced thermoplastic component 11 and has, for example, the same or substantially the same processing temperature.

The thermoplastic 12 is injected into the cavity 9 for the additional element 8 through at least one supply line in the mould 2, as shown in FIGS. 1 and 2. As a result of the temperature control of the fibre-reinforced thermoplastic component 11, the plastics matrix 5 thereof liquefies and the injected thermoplastic 12 penetrates the fibre arrangement 6 of the fibre-reinforced thermoplastic component 11 and fills the cavity 9, located on the other side, in the mould for the respective testing element 7. When the temperature of the fibre-reinforced thermoplastic component 11 in the mould 2 is adequately or suitably controlled and the temperature of an injected thermoplastic 12 is suitably controlled, the cavity 9 in the mould 2 for the respective testing element 7 can be completely filled with thermoplastic.

If, however, the temperature of the fibre-reinforced thermoplastic component 11 is not adequately controlled and/or the injected thermoplastic 12 is not suitably heated or temperature-controlled, the injected thermoplastic 12 cannot pass through the fibre arrangement 6 of the fibre-reinforced thermoplastic component 11, or can only pass through said arrangement in part, and accordingly cannot fill the cavity 9 for the testing element 7 or can only inadequately fill said cavity. As a result, the fibre-reinforced thermoplastic component assembly 1 does not have a testing element 7 or only has an incomplete testing element. Provided that the injected thermoplastic 12 was at a suitable processing temperature during processing, it can be concluded that the fibre-reinforced thermoplastic component 11 was not adequately heated or the temperature thereof was not suitably controlled. Accordingly, the strength of the connection between the additional element 8 and the fibre-reinforced thermoplastic component 11 of the fibre-reinforced thermoplastic component assembly 1 is sub-optimal or inadequate.

If, in addition, the thermoplastic of the testing element 7 is a different colour from the plastics matrix 5 of the thermoplastic component 11, it can also be determined whether or not the connection between the additional element 8 and the thermoplastic component 11 is as intended or is strong enough. If the temperature of the thermoplastic component 11 is suitably controlled, the plastics material of the plastics matrix 5 mixes with the plastics material of the testing element 7. The finished testing element 7 therefore has a different colouring and/or a different colour gradient than the thermoplastic originally injected for this purpose, since the two different colour plastics materials of the testing element 7 and the plastics matrix 5 mix together. If, however, the temperature of the thermoplastic component 11 is not suitably or adequately controlled, the plastics matrix 5 of the thermoplastic component 11 for example does not melt or does not melt to a sufficient extent to mix with the plastics material of the testing element 7, and therefore the testing element 7 is substantially the same colour as its injected thermoplastic. From this, it can in turn be concluded, for example, that the strength of the additional element 8 and the thermoplastic component 11 is insufficient. To determine the colour of the testing element 7, it is possible to use, for example, a visual assessment device, such as a sensor for determining the colour and/or at least one camera, or the human eye.

Moreover, the geometry of the testing element 7 etc. can be determined for example by means of a camera or another suitable device for determining the geometry of the testing element in order to check the formation of the testing element 7, and the geometry can be compared with a desired geometry in order to determine whether the testing element 7 has been completely or incompletely formed.

When completely formed, the testing element 7 can also independently or automatically actuate a trigger device (not shown), such as actuate a switch, interrupt a photoelectric sensor, etc. In this case, said trigger device can for example be used to enable a subsequent process, such as subsequent welding of sockets or another subsequent process. If the trigger device is actuated by the completely formed testing element 7, a subsequent process is started. If, however, the testing element 7 is not completely formed, it cannot actuate the trigger device, i.e. for example cannot actuate the switch or interrupt the photoelectric sensor, and accordingly no subsequent process is enabled.

As shown in the embodiment in FIGS. 1 and 2, the respective testing element 7 is arranged on the opposite side of the fibre-reinforced thermoplastic component 11 to the additional element 8. In this case, the testing element 7 can be arranged so as to overlap the additional element 8 either completely or at least in part, as shown in FIGS. 1 and 2, or arranged so as to be offset therefrom and so as not to overlap said additional element, as indicated by a dotted line in FIG. 1. The testing element 7 cannot only be arranged on the opposite side of the thermoplastic component 11 to the additional element 8, but rather can also be arranged on the same side of the thermoplastic component 11 as the additional element 8, as shown in FIGS. 3 and 4. If there are a plurality of testing elements 7, these can be arranged on the same side of the thermoplastic component 11 as the additional element 8 and/or on the opposite side of the thermoplastic component 11.

In addition, testing elements 7 are preferably arranged in a flow path of the injected thermoplastic 12. In the process, the thermoplastic 12 flows from the supply line 13 into the cavity 9 for the testing element 8 on one side of the fibre-reinforced thermoplastic component 11 and through the fibre arrangement 6 of the fibre-reinforced thermoplastic component 11 into the cavities 9 for the testing elements 7. In this case, the testing elements 7 are arranged on the opposite side of the fibre-reinforced thermoplastic component 11 or thermoplastic component assembly 1 to the additional element 8. The flow path is shown here by way of example in FIGS. 1 and 2 and indicated in a purely schematic manner by means of arrows.

Instead of injection moulding, the fibre-reinforced thermoplastic component 11 can also be provided with a testing element 7 by means of extrusion for example, in order to form a fibre-reinforced thermoplastic component assembly 1 according to an embodiment of the invention. However, the invention is not limited to injection moulding and extrusion.

As described above, a fibre-reinforced thermoplastic component 11 has reinforcing fibres which are provided with a thermoplastic matrix 5. In this case, the reinforcing fibres can, for example, be embedded in the thermoplastic matrix 5 and/or impregnated therewith. As described above, the fibres can, for example, form a fibre arrangement 6 in the form of at least one woven fabric, a fibre strand, a roving, a weft-knitted fabric, a braided fabric, a non-woven fabric, a mat and/or a fibrous web. In this context, the fabrics can be multiaxial fabrics or unidirectional fabrics, etc. In addition, the fibres can be short, long and/or continuous fibres.

Furthermore, the fibre-reinforced thermoplastic component can also for example be made of Twintex®. In this case, reinforcing fibres, such as glass fibres, are processed together with thermoplastic fibres, for example made of polypropylene, to form rovings, which are interweaved and then heated and shaped under pressure into a semi-finished product.

The fibrous material can, for example, be glass fibre, aramid fibre, carbon fibre, sisal, hemp, coconut fibre, cotton fibre and/or flax, etc. The invention is not limited to the above fibrous materials and the above types of fibre processing, such as a woven fabric, fibrous web, weft-knitted fabric, etc.

In this case, the fibre-reinforced thermoplastic component 11 as a planar semi-finished product, as shown in FIG. 1, or as a shaped structural component (not shown) can be provided with the at least one additional element 8 and with at least one testing element 7, in order to form a fibre-reinforced thermoplastic component assembly 1 according to the invention.

In this context, the fibre-reinforced thermoplastic component 11 to be encapsulated, as shown in FIGS. 1 and 2, can be additionally shaped therewith or deformed in the injection mould 2 for example in one operation (not shown). This has the advantage of reducing production costs. The fibre-reinforced thermoplastic component 11 can also be deformed for example prior to encapsulation or following encapsulation.

The additional element 8 is formed on a first side of the fibre-reinforced thermoplastic component 11 and the at least one testing element 7 is formed on the same side of the fibre-reinforced thermoplastic component 11 as the additional element 8 or on the opposite side thereto.

FIGS. 3 and 4 are both sectional views of a finished fibre-reinforced thermoplastic component assembly 1.

If, as shown in the sectional view in FIG. 3, the fibre-reinforced thermoplastic component 11 has been adequately heated, the injected plastics material flows through or penetrates the fibre-reinforced thermoplastic component 11 and its fibre arrangement 6, for example a fibre fabric or a nonwoven, as indicated previously by an arrow in FIG. 2, and completely fills the cavity in the mould for the testing element, for example together with the thermoplastic of the plastics matrix 5 of the fibre-reinforced thermoplastic component. A fibre-reinforced thermoplastic component assembly 1 is thus obtained having the additional element 8 on one side and the complete testing elements 7 on the other side, as shown in FIG. 3.

In addition, a testing element 7 can also be formed on the thermoplastic component 11 on the same side as the additional element 8 and optionally also as a part of the additional element 8, as indicated by a dashed line in FIGS. 3 and 4. In this case, the testing element 7 is for example arranged in the flow path of the injected thermoplastic such that the additional element 8 is first in the flow path and the testing element 7 follows thereafter. If the testing element 7 is no longer completely formed, as indicated by a dashed line in FIG. 4, it can thus be concluded that the strength of the connection between the additional element 8 and the thermoplastic component is inadequate. Conversely, if the testing element 7 is completely formed, as indicated in FIG. 3 by a dashed line, it can thus be concluded that the strength of the connection between the additional element 8 and the thermoplastic component is adequate.

In this case, a visual check, for example by means of sight and/or by a camera, as to whether the testing element 7 has been completely formed can be carried out in a very simple manner. It can now be deduced therefrom that the fibre-reinforced thermoplastic component was adequately heated previously, and therefore the strength of the connection between the additional element 8, for example a rib, and the fibre-reinforced thermoplastic component of the thermoplastic component assembly 1 is adequate. In this case, there is no need for a destructive test to test the adequate strength of the connection.

If, however, the fibre-reinforced thermoplastic component, as shown in the sectional image of a fibre-reinforced thermoplastic component assembly 1 in FIG. 4, has not been adequately or sufficiently heated and/or if the organic sheet cools down too much as a result of contact with the tool or excessive handling time, the injected thermoplastic does not penetrate or does not adequately penetrate the fibre arrangement 6, for example a woven or nonwoven fabric, of the fibre-reinforced thermoplastic component, and therefore the cavity in the mould for the testing element cannot be completely filled with plastics material. A fibre-reinforced thermoplastic component assembly 1 is thus obtained, as shown in FIG. 4, which is formed having the additional element 8 but which does not have any testing elements 7 or only has incompletely formed testing elements, including the testing element 7 which is only incompletely formed in the region of the additional element 8 and which is marked by a dashed line in FIG. 4.

In this case, a visual check, for example by means of sight and/or by a camera etc., as to whether the testing element 7 has not been formed or has only been incompletely formed can be carried out in a very simple manner. It can now be deduced therefrom that the fibre-reinforced thermoplastic component was inadequately heated previously, and therefore the strength of the connection between the additional element 8, for example a rib, and the fibre-reinforced thermoplastic component is inadequate or insufficient. There is no need for a destructive test of the fibre-reinforced thermoplastic component assembly 1 to test the strength of the connection which is insufficient in the present case.

FIG. 5 shows an embodiment of a fibre-reinforced thermoplastic component assembly 1 from the side on which a plurality of testing elements 7 are intended to be formed on the fibre-reinforced thermoplastic component assembly 1. As can be seen in FIG. 5, however, testing elements 7 could not be formed at the points marked by the arrows. Thermoplastic was not able to adequately penetrate the fibre arrangement 6 (a fibre fabric in this case). It can be concluded therefrom that the strength of the connection between one of the additional components formed on the other side of the fibre-reinforced thermoplastic component assembly 1, for example a rib, a fastener, a cable guide, etc., and the fibre-reinforced thermoplastic component is inadequate. For this purpose, there is no need for a destructive test, such as a tension test, etc., or a CT analysis.

FIG. 6 shows an additional embodiment of a fibre-reinforced thermoplastic component assembly 1 from the side on which a plurality of testing elements 7 are formed on the fibre-reinforced thermoplastic component assembly 1. As can be seen in FIG. 6, testing elements 7 were able to be completely formed at the points marked by the arrows. It can be concluded from this that the strength of the connection between one of the additional components formed on the other side of the fibre-reinforced thermoplastic component assembly 1, for example a rib, a fastener, a cable guide, etc., and the fibre-reinforced thermoplastic component is adequate. There is again no need in this case for a destructive test, such as a tension test, etc., or a CT analysis.

In the embodiment shown in FIG. 6, the testing elements 7 have, for example, an additional function and accordingly form a functional element. The testing elements 7 in the embodiment in FIG. 6 are simultaneously formed as additional reinforcing ribs 10 or stiffening ribs. The testing elements 7 can also be formed as a clip, for example for cabling, as mounts, fasteners, cable guides, etc. In this context, the invention is not limited to the above examples for testing elements 7. The testing elements 7 can also be formed to have no additional function, for example as melt islands (not shown). Owing to their simple shape, melt islands of this type, for example in the form of curved projections as shown in FIG. 1-4, can be produced in a simple and cost-effective manner and are easy to inspect visually.

FIG. 7 is a sectional view through a mould 2 and a thermoplastic component 11 received therein, following the injection of plastics material to form a thermoplastic component assembly 1 having at least one additional element 8 and at least one testing element 7.

The mould 2, in the form of a tool, comprises a male mould 14 and a female mould 15. The thermoplastic component 11 is heated to the processing temperature of its plastics matrix and placed in the mould 2. When the thermoplastic components 11 have thin walls, heating in the tool or mould 2 is also possible. The mould 2 or its male mould 14 and female mould 15 are then closed and the plasticised additive, i.e. the thermoplastic, is injected. The injection point 16 for the thermoplastic for the additional element 8 is selected, for example, at the uppermost and outermost point of the additional element 8. However, any other position can also be selected for the injection point 16, and a plurality of injection points can be provided rather than just one.

The cavity or hollow space in the mould 2 for the additional element 8 is filled and the material then has to flow further into the testing element 7. This only happens if the temperature of the fibre-reinforced thermoplastic component 11, for example an organic sheet, is still above the crystallisation limit. In the thermoplastic component assembly 1 shown in FIG. 7 and then in FIGS. 8 and 9, a plurality of testing elements 7 are provided. As shown in FIG. 7, a testing element 7 is arranged on the thermoplastic component 11 on the same side as the additional element 8 and such that the thermoplastic injected at the injection point 16 has to cover a flow path towards the testing element 7 that is at least the same as or larger than the flow path towards the additional element 8.

FIG. 8 is a perspective view and FIG. 9 is a plan view, respectively, of the finished thermoplastic component assembly 1, once the finished thermoplastic component assembly 1 has been removed from the mould 2 in FIG. 7. The thermoplastic component assembly 1 comprises for example a rib as an additional element 8, and for example three testing elements 7 provided on the thermoplastic component 11 of the thermoplastic component assembly 1 on the same side as the additional element 8.

Here, the testing elements 7 are arranged such that the thermoplastic injected at the injection point 16 has to cover a flow path towards the testing element 7 that is at least the same as or larger than the flow path towards the additional element 8. In this case, a testing element 7 is formed as part of the additional element 8 and thus forms a part of the rib. As shown previously in FIG. 7 and indicated by a dashed line in FIG. 8, the injection point or moulding point 16 for the thermoplastic can in this context be provided at the uppermost and outermost point of the additional element 8. Alternatively, the injection point or moulding point 16 for the thermoplastic can also be provided, for example, at the lowest and outermost point of the additional element 8. The testing element 7 as part of the additional element 8 can in this case be provided, for example, at the other end of the additional element 8 at the uppermost and outermost point of the additional element 8 and/or at the lowest and outermost point of the additional element 8, as indicated by a dotted line in FIG. 8.

In principle, any other position can be provided for the testing element and the injection point or moulding point 16; the view in the figures is merely an example and the invention is not limited thereto.

By means of embodiments of the invention, it is very easy to recognise on the finished component assembly whether the temperature of the fibre-reinforced thermoplastic component had been sufficiently or adequately controlled. For this purpose, as described previously with reference to FIGS. 1 and 6, the testing element 7 provided on the fibre-reinforced thermoplastic component assembly 1 is preferably provided on the opposite side of the thermoplastic component or thermoplastic component assembly to the injection or moulding of plastics material to the fibre-reinforced thermoplastic component. In this context and as described above, a melt island or for example an additional functional element, such as a rib, a clip, a mount and/or a guide, etc. can be attached to the fibre-reinforced thermoplastic component of the thermoplastic component assembly 1 as a testing element 7.

The testing element 7, for example in the form of a melt island or a functional element, can be assessed visually, for example by a visual check and/or by means of a camera, etc. The invention is not limited to the above visual assessments. In principle, it is possible to provide any type of visual check or a different test that is suitable for determining whether the testing element 7 has been formed completely, partly or not at all.

In further embodiments of the invention, one or more testing elements 7 are provided at the points on a fibre-reinforced thermoplastic component which require a defined flow path for the injected melt. FIG. 1 shows an example of a flow path of this type. In this context, the testing elements 7 can be arranged along this flow path.

Advantages of embodiments of the invention are that a simple and non-destructive quality control check for the component assembly, for example consisting of continuous-fibre-reinforced thermoplastic polymer, can be inspected at any time on the component assembly. At least one additional element can be embedded or worked into the fibre-reinforced thermoplastic component assembly as required, for example a profile made of metal or of another material or of a combination of materials

It is also possible to monitor a number of critical points on large component assemblies. As a result, the respective testing element 7 can form, for example, a quality assurance feature at no extra cost when integration parts are used, in particular when the testing element 7 is also formed as a functional element, for example a reinforcing rib, a mount, a guide, etc.

Although the present invention has been fully described above by means of preferred embodiments, it is not limited thereto, but rather may be modified in a number of ways. In particular, the embodiments described above, particularly individual features thereof, can also be combined with one another.

### LIST OF REFERENCE NUMERALS

1 fibre-reinforced thermoplastic component assembly
2 mould
3 first mould half
4 second mould half
5 plastics matrix
6 fibre arrangement
7 testing element
8 additional element
9 cavity
10 reinforcing rib
11 fibre-reinforced thermoplastic component
12 plastics material to be injected
13 supply line
14 male mould
15 female mould
16 injection point

1. Fibre-reinforced thermoplastic component assembly comprising
a fibre-reinforced thermoplastic component
having a first side and a second side opposite the first side,
at least one additional element which is made of a thermoplastic and is arranged on the first side of the fibre-reinforced thermoplastic component, and
at least one testing element which is made of a thermoplastic, is arranged on the first or second side of the fibre-reinforced thermoplastic component so as to be visible, and is designed for testing the connection between the additional element and the fibre-reinforced thermoplastic component.

2. Assembly according to claim 1,
characterised in that
the at least one testing element is arranged on the second side of the fibre-reinforced thermoplastic component so as to not overlap the additional element or so as to overlap the additional element at least in part.

3. Assembly according to claim 1,
characterised in that
the at least one testing element is arranged on the at least one additional element as an additional portion.

4. Assembly according to claim 1,
characterised in that
a different colour thermoplastic in each case is used to form the at least one testing element, the at least one additional element and/or the thermoplastic component.

5. Assembly according to claim 1,
characterised in that
the at least one testing element and/or the at least one additional element are formed on the fibre-reinforced thermoplastic component by means of injection moulding or by extrusion.

6. Assembly according to claim 1,
characterised in that
the at least one testing element is formed as a melt island.

7. Assembly according to claim 1,
characterised in that
the at least one testing element is formed as a functional element, the functional element being a rib, a mount, a guide, a reinforcement, a stiffener and/or a clip.

8. Assembly according to claim 1,
characterised in that
the at least one testing element is arranged on the fibre-reinforced thermoplastic component assembly so as to be visible to the visual assessment device so that the testing element can be visually assessed by means of an visual assessment device, in particular by means of at least one camera and at least one sensor for detecting the colour, in particular for assessing the geometry and/or the colour of the at least one testing element.

9. Assembly according to claim 1,
characterised in that
the at least one testing element is arranged on the fibre-reinforced thermoplastic component assembly so as to be visible to the eye of a user so that the testing element can be assessed by means of a visual check.

10. Assembly according to claim 1,
characterised in that
a plurality of testing elements are provided on the thermoplastic component assembly along a flow path of a thermoplastic supplied to the fibre-reinforced thermoplastic component.

11. Assembly according to claim 1,
characterised in that
the at least one additional element is a rib, a mount, a guide, a reinforcement, a stiffener and/or a clip.

12. Assembly according to claim 1,
characterised in that
the fibre-reinforced thermoplastic component assembly consists of a fibre arrangement consisting of glass fibres, aramid fibres, carbon fibres, sisal, hemp, coconut fibres, cotton fibres and/or flax, and the fibre arrangement forms at least one woven fabric, a fibre strand, a roving, a weft-knitted fabric, a braided fabric, a nonwoven fabric, a mat and/or a fibrous web.

13. Assembly according to claim 1,
characterised in that
the thermoplastic component is a continuous-fibre-reinforced thermoplastic component.

14. Assembly according to claim 1,
characterised in that
the at least one testing element is provided on the fibre-reinforced thermoplastic component assembly such that, when said testing element is completely formed, it autonomously or automatically actuates a trigger device, in particular a switch and/or a photoelectric sensor, and when it is not formed or is incompletely formed, it does not actuate the trigger device.

15. Assembly according to claim 1,
characterised in that
the at least one testing element is arranged such that said element is completely formed when the temperature of the thermoplastic component was above the limit temperature for an integral bond with the injected thermoplastic during the injection moulding of the additional element with the thermoplastic, and is not formed or is incompletely formed when the temperature of the thermoplastic component was below the limit temperature for an integral bond with the injected thermoplastic during the injection moulding of the additional element with the thermoplastic.

16. Method for producing a fibre-reinforced thermoplastic component assembly, comprising the steps of:
providing a fibre-reinforced thermoplastic component having a first side and a second side opposite the first side,
forming, from a thermoplastic, at least one additional element on the first side of the fibre-reinforced thermoplastic component; and
forming, from a thermoplastic, at least one testing element on the first or second side of the fibre-reinforced thermoplastic component, wherein the at least one testing element is arranged on the fibre-reinforced thermoplastic component as to be visible and is designed for testing the connection between the additional element and the fibre-reinforced thermoplastic component.

17. Method according to claim 16,
characterised in that
the at least one additional element and the at least one testing element are formed on the fibre-reinforced thermoplastic component by injection moulding or by extrusion.

18. Method according to claim 16,
characterised in that
the fibre-reinforced thermoplastic component is deformed before, during and/or after the step of forming at least one additional element and/or at least one testing element on the fibre-reinforced thermoplastic component.
